(19) United States
(12) Patent Application Publication
(10) Pub. No.: US 2023/0165875 A1
(43) Pub. Date: Jun. 1, 2023

(54) METHYLTHIONINIUM COMPOUNDS FOR USE IN THE TREATMENT OF COVID-19

(71) Applicant: WisTa Laboratories Ltd., Singapore (SG)

(72) Inventors: Claude Michel Wischik, Aberdeen, Aberdeenshire (GB); Mohammad Arastoo, Aberdeen, Aberdeenshire (GB); Michael Philip Mazanetz, Newlands, Glasgow (GB)

(73) Assignee: WisTa Laboratories Ltd., Singapore (SG)

(21) Appl. No.: 17/997,758

(22) PCT Filed: Apr. 30, 2021
(86) PCT No.: PCT/EP2021/061480

§ 371 Date: Nov. 2, 2022

(30) Foreign Application Priority Data
May 5, 2020 (GB) 2006659.3
Oct. 26, 2020 (GB) 2016955.3

(51) Int. Cl. A61K 31/54; A61K 45/06; A61K 47/02

(52) U.S. Cl. CPC A61K 31/54; A61K 45/06; A61K 47/02

(57) ABSTRACT

The present invention provides methods of treating COVID-19 in a subject using methylthioninium compounds.

### TECHNICAL FIELD

The present invention relates generally to methods and materials for use in the treatment of COVID-19.

### BACKGROUND ART

The novel coronavirus disease 2019 (COVID-19) caused by Severe Acute Respiratory Syndrome Coronavirus 2 (SARS-CoV-2) poses a major healthcare and economic threat globally. Although most infections are self-limited, according to current estimates at the time of filing about 14% of infected patients have severe disease and require hospitalisation, 5% of infected patients have very severe conditions and require intensive care admission (mostly for ventilation) and 4% of infected patients die (WHO, 2020).

The prospects for a return to socioeconomic normality are critically dependent on the development of new treatment approaches.

Whilst it is hoped that the development of a vaccine will provide a preventative strategy, vaccines may still be non-optimal for reasons of potentially resistant viral mutations, toxicity, and problems with treatment of long-lasting functional impairments.

Therefore, even when/if a vaccine is developed, there is a need for adjunctive therapeutic approaches which can mitigate the worst effects of the infection both in severity and duration.

A recent WHO-sponsored study in over 11,000 subjects in over 400 hospitals in 30 countries found that none of the 4 treatments evaluated (remdesivir, hydroxychloroquine, lopinavir/ritonavir and interferon) had any effect on overall mortality, initiation of ventilation or duration of hospital stay in hospitalized patients (WHO Solidarity Trial Consortium, 15 Oct. 2020).

Repositioning of known drugs can significantly accelerate the development and deployment of therapies for COVID-19 and therefore there is an interest in profiling known drugs which may inhibit viral replication. For example Riva et al. (“A Large-scale Drug Repositioning Survey for SARS-CoV-2 Antivirals.” bioRxiv (2020)) profiled approximately 12,000 clinical-stage or FDA approved small molecules and reported the identification of 30 known drugs that inhibited viral replication under the tested conditions, of which six were characterized for cellular dose-activity relationships, and showed effective concentrations which they believed to be likely to be commensurate with therapeutic doses in patients. These include the PlKfyve kinase inhibitor Apilimod, cysteine protease inhibitors MDL-28170, Z LVG CHN2, VBY-825, and ONO 5334, and the CCR1 antagonist MLN-3897.

However screening of this type focusses on only a single attribute of SARS-CoV-2 (here: viral replication in Vero E6 cells) and the concentration of compound used in the screen (here: 5 μM) may not be optimal for detecting all promising candidates, or predictive of appropriate in vivo therapeutic doses.

Furthermore COVID-19 has been reported to be particularly harmful in vulnerable patients such as the elderly. Many potential therapeutics may not be suitable for use in that patient group.

Thus it can be seen that providing compounds or combinations of compounds which can be used safely in an elderly population, can target multiple attributes of the COVID-19 aetiology, and providing dosage information applicable to that, provides a useful contribution to the art.

### DISCLOSURE OF THE INVENTION

The present invention provides for the use of certain hydromethylthionine salts (referred to as “LMTX” below) as a monotherapy or combination therapy for the treatment of COVID-19. In the light of the disclosure herein, it can be expected that such treatment can provide a number of beneficial treatment effects.

Based on proprietary pharmacokinetic studies the present inventors define dosages of LMTX which can be expected to achieve in vivo levels in tissues which will achieve significant reductions in SARS-CoV-2 toxicity, and other benefits described herein.

WO2007/110627 disclosed certain 3,7-diamino-10H-phenothiazinium salts, effective as drugs or pro-drugs for the treatment of diseases including Alzheimer's disease and other diseases such as Frontotemporal dementia (FTD), as well as viral diseases generally. These compounds are also in the “reduced” or “leuco” form when considered in respect of MTC. These leucomethylthioninium compounds were referred to herein as “LMTX” salts.

WO2012/107706 described other LMTX salts having superior properties to the LMTX salts listed above, including leuco-methylthioninium bis(hydromethanesulfonate) (LMTM) (WHO INN designation: hydromethylthionine):

| | |
|---|---|
| 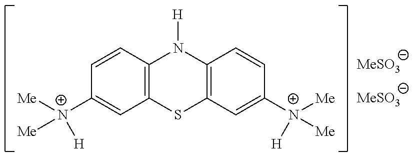 | N,N,N′,N′-tetramethyl-10H- phenothiazine-3,7- diaminium bis(hydromethanesulfonate) LMT•2MsOH/LMTM |

These publications described LMTX in general terms for treatment of viral disease but not for the treatment of COVID-19 or other coronaviruses, specifically.

MTC (methylthioninium chloride, methylene blue) is an FDA and EMA approved drug with a long history of clinical use. MTC and is currently being investigated to assess its potential utility as an antiviral drug against SARS-CoV-2 (see Reference Example 1.)

LMTX delivers the same MT (methylthionine) moiety systemically, but is more suitable for oral and intravenous use than MTC as it has improved absorption, red cell penetration and deep compartment distribution (Baddeley et al., 2015). LMTX can be used at a substantially lower dose than MTC and is thus better tolerated.

Independently of MTC, the antimalarial compound chloroquine and the related hydroxychloroquine are currently being investigated globally to assess their effectiveness as antiviral drugs against SARS-CoV-2.

However, chloroquine has a narrow therapeutic ratio such that significant electrophysiological effects occur at plasma concentrations approaching the micromolar range which is required for pharmacological activity. A Brazilian trial of chloroquine diphosphate for COVID-19 cases at two doses (https://doi.org/10.1101/2020.04.07.20056424) was reportedly halted because of cardiac deaths.

LMTX has a more benign safety profile. The inventors have established that LMTX does not demonstrate cardiotoxicity.

The present specification discloses that not only can LMTX provide benefits to subjects in permitting reduction of viral toxicity, but additionally:
LMTX can enhance mitochondrial function; there is mounting evidence to suggest a link between COVID-19 and mitochondrial dysfunction.
LMTX can enhance blood oxygen capacity, as evidence in clinical trials performed by the present inventors. COVID-19 has been associated with the emergence of both methemoglobinemia and hypoxaemia in patients.
LMTX may also improve CNS sequelae of COVID-19. Several reports indicate that COVID-19 may have detrimental effects on the central nervous system.

Thus in one aspect there is disclosed a method of therapeutic treatment of COVID-19 in a subject,
which method comprises administering to said subject a methylthioninium (MT)-containing compound,
wherein said administration provides a total daily oral dose of between more than 30 mg to 250 mg of MT to the subject per day, optionally split into 2 or more doses,
or wherein said administration provides a total daily intravenous (IV) dose of between 10 and 200 mg of MT to the subject per day,
wherein the MT-containing compound is an LMTX compound of the following formula:

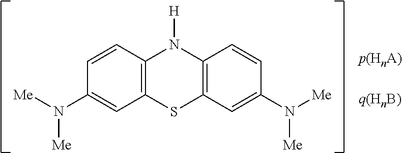

wherein each of HₙA and HₙB (where present) are protic acids which may be the same or different,

and wherein p=1 or 2; q=0 or 1; n=1 or 2; (p+q)×n=2,

or a hydrate or solvate thereof.

In one embodiment the subject is a human who has been diagnosed as having COVID-19. The method may comprise making said diagnosis.

In one aspect there is disclosed a method of prophylactic treatment of COVID-19 in a subject,
which method comprises administering to said subject a methylthioninium (MT)-containing compound,
wherein the MT-containing compound is an LMTX compound as defined above, or a hydrate or solvate thereof.

In one embodiment the subject is a human who has been assessed as having suspected or probable COVID-19 e.g. a subject who has been in close contact with one or more COVID-19 cases; a subject who is at least 65 years old; a subject living in a nursing home, care home, or long-term care facility; a subject with a relevant underlying medical condition.

Preferably said administration provides a total daily oral dose of more than 35, 40, 50, or 60 mg and less than or equal to 250 mg of MT to the subject per day, optionally split into 2 or more doses.

The total daily oral dose may be greater than or equal to 30.5, 30.6, 31, 35, 37.5, 40, 45, 50, 55, 60, 65, 70, 75, 80, 85, 90, 95, 100, 105, 110, 115, 120, 125, or 130 mg.

The total daily oral dose is preferably greater than or equal to 30.5, 30.6, 30.7, 30.8, 30.9, or 31 mg.

The total daily oral dose may be 60, 75, or 120 mg.

The total daily dose of the compound may be administered as a split dose twice a day or three times a day.

As explained below, when administering the MT dose split in a larger number of doses/day it may be desired to use a smaller total amount within the recited range, compared to a single daily dosing, or a smaller number of doses per day.

When intravenous doses are used in the present invention, said intravenous administration provides a total daily intravenous (IV) dose of 10 and 200 mg of MT to the subject per day.

The range of 10 and 200 mg encompasses those dosages that are expected to achieve an appropriate in reduction in toxicity as explained hereinafter whether administrated by continuous infusion or on the basis of a reasonable number of spaced bolus dosages (e.g. 4 or more) in which case typically slightly higher total dosages are required to achieve the same effect as continuous dosing. The bolus itself may be administered over a short period appropriate to the volume, flow rate and concentration of drug in question e.g. 3 to 10 minutes, e.g. 5 minutes.

The Examples herein show equivalent dosages for continuous dosing, and IV bolus infusion administered 6-hourly 4 times per day (“iv q 6 hr”). Based on the disclosure herein equivalent dosages for bolus and continuous administration can be inferred.

For example a range of 17 to 122 mg/day continuous equates to 21 to 200 mg/day iv q 6 hr. In some embodiments the IV dosing is equivalent to these ranges.

In other embodiments said intravenous administration provides a total daily dose of between 30 and 150 mg of MT to the subject per day.

In other embodiments said intravenous administration provides a total daily dose of between 26 and 150 mg of MT to the subject per day.

In other embodiments said intravenous administration provides a total daily dose of between 26 and 148 mg of MT to the subject per day.

In other embodiments said intravenous administration provides a total daily dose of between 30 and 122 mg of MT to the subject per day by continuous dosing.

In other embodiments said intravenous administration provides a total daily dose of between 36 and 148 mg of MT to the subject per day by bolus dosing e.g. by iv q 6 hr.

In some embodiments, the IV dosage is:

About, 10, 11, 12, 13, 14, 15, 16, 17, 18, 19, 20, 25, 30, 35, 40, 45, 50, 60, 70, 80, 90, 100, 110, 120, 130 mg/day by continuous dosing.

About, 20, 25, 30, 35, 40, 45, 50, 60, 70, 80, 90, 100, 110, 120, 130, 140, 150, 160, 170, 180, 190, 200 mg/day by bolus dosing e.g. by iv q 6 hr or every 8 hr or 12 hr.

About, 30, 35, 40, 45, 50, 60, 70, 80, 90, 100, 110, 120, 125, 130 mg/day by continuous dosing.

About, 35, 40, 45, 50, 60, 70, 80, 90, 100, 110, 120, 130, 140, 150, 160, 170, 180, 190, 200 mg/day by bolus dosing e.g. by iv q 6 hr.

LMTX Compounds

Preferably the LMT compound is an “LMTX” compound of the type described in WO2007/110627 or WO2012/107706.

Thus the compound may be selected from compounds of the following formula, or hydrates or solvates thereof:

| | |
|---|---|
| 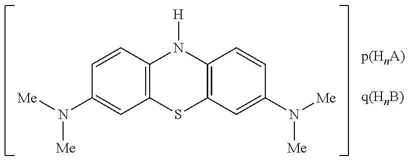 | Options: p = 1, 2 q = 0, 1 n = 1, 2 (p + q) × n = 2 |

Each of HₙA and HₙB (where present) are protic acids which may be the same or different.

By “protic acid” is meant a proton (H⁺) donor in aqueous solution. Within the protic acid A⁻ or B⁻ is therefore a conjugate base. Protic acids therefore have a pH of less than 7 in water (that is the concentration of hydronium ions is greater than 10⁻⁷ moles per litre).

In one embodiment the salt is a mixed salt that has the following formula, where HA and HB are different mono-protic acids:

| | |
|---|---|
| 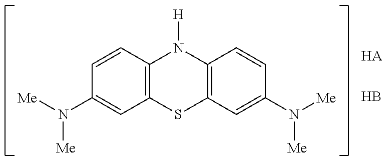 | when: p = 1 q = 1 n = 1 (1 + 1) × 1 = 2 |

However preferably the salt is not a mixed salt, and has the following formula:

| | |
|---|---|
| 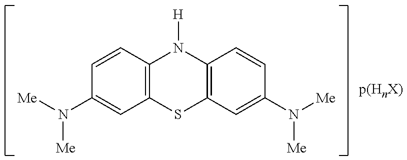 | when: p = 1, 2 n = 1, 2 p × n = 2 |

wherein each of HₙX is a protic acid, such as a di-protic acid or mono-protic acid.

In one embodiment the salt has the following formula, where H₂A is a di-protic acid:

| | |
|---|---|
| 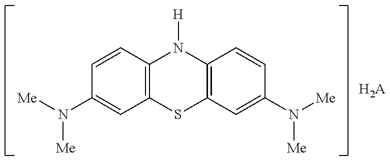 | when: p = 1 q = 0 n = 2 (1 + 0) × 2 = 2 |

Preferably the salt has the following formula which is a bis monoprotic acid:

| | |
|---|---|
| 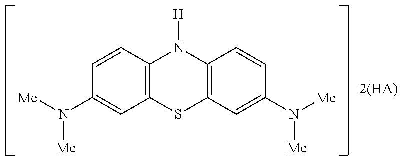 | when: p = 2 q = 0 n = 1 (2 + 0) × 1 = 2 |

Examples of protic acids which may be present in the LMTX compounds used herein include:

Inorganic acids: hydrohalide acids (e.g., HCl, HBr), nitric acid (HNO₃), sulphuric acid (H₂SO₄)

Organic acids: carbonic acid (H₂CO₃), acetic acid (CH₃COOH), methanesulfonic acid, 1,2-ethanedisulfonic acid, ethansulfonic acid, naphthalenedisulfonic acid, p-toluenesulfonic acid,

Preferred acids are monoprotic acid, and the salt is a bis(monoprotic acid) salt.

A preferred MT compound is LMTM:

| | | | |
|---|---|---|---|
| 1 | 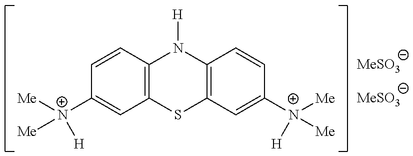 | LMT•2MsOH (LMTM) | 477.6 (1.67) |

Weight Factors

The anhydrous salt has a molecular weight of around 477.6. Based on a molecular weight of 285.1 for the LMT core, the weight factor for using this MT compound in the invention is 1.67. By “weight factor” is meant the relative weight of the pure MT-containing compound vs. the weight of MT which it contains.

Other weight factors can be calculated for example MT compounds herein, and the corresponding dosage ranges can be calculated therefrom.

Therefore the invention embraces a total daily dose of at least 50 mg of LMTM.

Other example LMTX compounds are as follows. Their molecular weight (anhydrous) and weight factor is also shown:

| | | | |
|---|---|---|---|
| 2 | 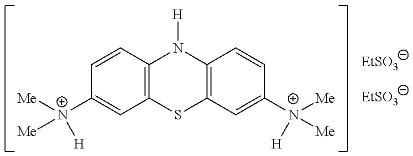 | LMT•2EsOH | 505.7 (1.77) |
| 3 | 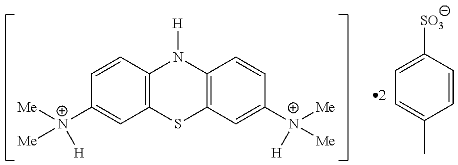 | LMT•2TsOH | 629.9 (2.20) |
| 4 | 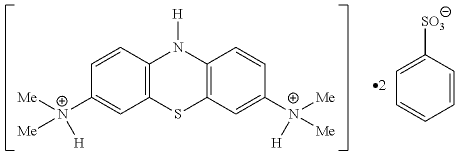 | LMT•2BSA | 601.8 (2.11) |
| 5 | 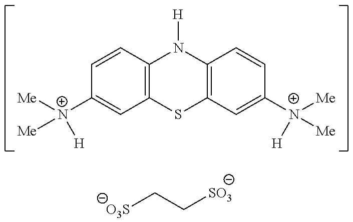 | LMT•EDSA | 475.6 (1.66) |
| 6 | 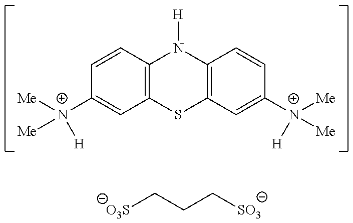 | LMT•PDSA | 489.6 (1.72) |
| 7 | 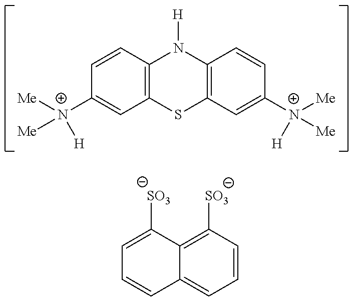 | LMT•NDSA | 573.7 (2.01) |
| 8 | 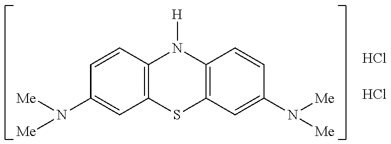 | LMT•2HCl | 358.33 (1.25) |

The dosages described herein with respect to MT thus apply mutatis mutandis for these MT-containing compounds, as adjusted for their molecular weight.

Accumulation Factors

As will be appreciated by those skilled in the art, for a given daily dosage, more frequent dosing can lead to greater accumulation of a drug.

Therefore in certain embodiments of the claimed invention, the total daily dosed amount of MT compound may be relatively lower, when dosing more frequently (e.g. twice a day [bid] or three times a day [tid]), or higher when dosing once a day [qd].

Treatment and Prophylaxis

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition.

The term “therapeutically-effective amount,” as used herein, pertains to that amount of a compound of the invention, or a material, composition or dosage from comprising said compound, which is effective for producing some desired therapeutic effect, commensurate with a reasonable benefit/risk ratio, when administered in accordance with a desired treatment regimen. The present inventors have demonstrated that a therapeutically-effective amount of an MT compound in respect of the diseases of the invention can be much lower than was hitherto understood in the art.

The invention also embraces treatment as a prophylactic measure.

The term “prophylactically effective amount,” as used herein, pertains to that amount of a compound of the invention, or a material, composition or dosage from comprising said compound, which is effective for producing some desired prophylactic effect, commensurate with a reasonable benefit/risk ratio, when administered in accordance with a desired treatment regimen.

“Prophylaxis” in the context of the present specification should not be understood to circumscribe complete success i.e. complete protection or complete prevention. Rather prophylaxis in the present context refers to a measure which is administered in advance of a condition, or prior to the worsening of such a condition, with the aim of preserving health by helping to delay, mitigate or avoid that particular condition.

Combination Treatments and Monotherapy

The term “treatment” includes “combination” treatments and therapies, in which two or more treatments or therapies for COVID-19 are combined, for example, sequentially or simultaneously. These may be symptomatic or disease modifying treatments.

The particular combination would be at the discretion of the physician.

In combination treatments, the agents (i.e., an MT compound as described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

An example of a combination treatment of the invention would be wherein the LMTX treatment is combined with an anti-inflammatory such as dexamethasone.

Another combination treatment is with chloroquine or hydroxychloroquine. Suggested protocols recommended for SARS-CoV-2 infection include a loading dose of 400 mg twice daily of hydroxychloroquine sulfate given orally, followed by a maintenance dose of 200 mg given twice daily for 4 days. An alternative is chloroquine phosphate when given 500 mg twice daily 5 days in advance (see e.g. Yao et al “In Vitro Antiviral Activity and Projection of Optimized Dosing Design of Hydroxychloroquine for the Treatment of Severe Acute Respiratory Syndrome Coronavirus 2 (SARS-CoV-2)” Clinical Infectious Diseases, 2020, Mar. 9.

The MT-containing compound and the second agent may be administered sequentially within 12 hours of each other, or the subject may be pre-treated with one for a sustained period prior to treatment with the other, or the agents may be administered simultaneously, optionally within a single dosage unit.

As described herein, in relation to combination therapies, the invention provides methods of enhancing the therapeutic effectiveness of a first compound which is an MT compound at a dose described herein for the treatment of COVID-19, the method comprising administering to the subject a second agent as described herein.

The invention further provides a first compound which is an MT compound at a dose described herein in a method of treatment of COVID-19 in a subject in a treatment regimen which additionally comprises treatment with a second agent.

The invention further provides use of a second agent to enhance the therapeutic effectiveness of an MT compound at a dose described herein in the treatment of COVID-19 in the subject.

The invention further provides an MT compound at a dose described herein and a second agent for use in a combination method of the invention.

The invention further provides a second agent for use in a method of enhancing the therapeutic effectiveness of an MT compound at a dose described herein in the treatment of COVID-19 in a subject.

The invention further provides use of a first compound which is an MT compound at a dose described herein in combination with a second agent, in the manufacture of a medicament for treatment of COVID-19.

The invention further provides use of an MT compound at a dose described herein in the manufacture of a medicament for use in the treatment of COVID-19, which treatment further comprises use of a second agent.

The invention further provides use of a second agent, in the manufacture of a medicament for use in the treatment of COVID-19 in a subject, which treatment further comprises use of an MT compound at a dose described herein and COVID-19.

Second agent for use in combination treatments include one or more of:

chloroquine or hydroxychloroquine; lopinavir-ritonavir; arbidol; azithromycin, remdesivir, favipiravir, anti-inflammatory treatments such as actemra (tocilizumab), corticosteroids such as dexamethasone; convalescent plasma; (see e.g. Thorlund, Kristian, et al. “A real-time dashboard of clinical trials for COVID-19.” The Lancet Digital Health (2020); a SARS-CoV-2-neutralising antibodies (see Kreer, Christoph, et al. “Longitudinal isolation of potent near-germline SARS-CoV-2-neutralizing antibodies from COVID-19 patients.” Cell 182.4 (2020): 843-854.)

In other embodiments the treatment is a “monotherapy”, which is to say that the MT-containing compound is not used in combination (within the meaning discussed above) with another active agent for treating COVID-19 in the subject.

Duration of Treatment

For treatment of COVID-19, a treatment regimen based on the MT compounds described herein will preferably extend over a sustained period of time appropriate to the disease and symptoms. The particular duration would be at the discretion of the physician.

For example, the duration of treatment may be:

1 to 14, e.g. 1, 2, 3, 4, 5, 6, 7, 8, 9, 10, 11, 12, 13 or 14 days.

1 to 4, e.g. 1, 2, 3 or 4 weeks.

For prophylaxis, the treatment may be ongoing.

In all cases the treatment duration will generally be subject to advice and review of the physician.

Pharmaceutical Dosage Forms

The MT compound of the invention, or pharmaceutical composition comprising it, may be administered to the stomach of a subject/patient orally (or via a nasogastric tube) or intravenously.

Typically, in the practice of the invention the compound will be administered as a composition comprising the compound, and a pharmaceutically acceptable carrier or diluent.

In some embodiments, the composition is a pharmaceutical composition (e.g., formulation, preparation, medicament) comprising a compound as described herein, and a pharmaceutically acceptable carrier, diluent, or excipient.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

In some embodiments, the composition is a pharmaceutical composition comprising at least one compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In some embodiments, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, N.Y., USA), Remington's Pharmaceutical Sciences, 20th edition, pub. Lippincott, Williams & Wilkins, 2000; and Handbook of Pharmaceutical Excipients, 2nd edition, 1994.

One aspect of the present invention utilises a dosage unit (e.g., a pharmaceutical tablet or capsule) comprising an MT compound as described herein (e.g., obtained by, or obtainable by, a method as described herein; having a purity as described herein; etc.), and a pharmaceutically acceptable carrier, diluent, or excipient.

The “MT compound”, although it may be present in relatively low amount, is the active agent of the dosage unit, which is to say is intended to have the therapeutic or prophylactic effect in respect of COVID-19. Rather, the other ingredients in the dosage unit will be therapeutically inactive e.g. carriers, diluents, or excipients.

Thus, preferably, there will be no other active ingredient in the dosage unit, no other agent intended to have a therapeutic or prophylactic effect in respect of a disorder for which the dosage unit is intended to be used, other than in relation to the combination treatments described herein.

In some embodiments, the dosage unit is a tablet.

In some embodiments, the dosage unit is a capsule.

In some embodiments, the dosage unit is provided as a syrup.

In some embodiments, said capsules are gelatine capsules.

In some embodiments, said capsules are HPMC (hydroxypropylmethylcellulose) capsules.

The appropriate quantity of MT in the composition will depend on how often it is taken by the subject per day, or how many units are taken at one time. Therefore dosage units may individually contain less than the total daily dose.

An example dosage unit may contain 10 to 250 mg of MT.

In some embodiments, the amount is about 10, 11, 12, 13, 14, 15, 16, 17, 18, 19, 20, 30, 40, 50, 60, 70, 80, 90, 100, 110, 120, 130, 140, 150 mg of MT.

Using the weight factors described or explained herein, one skilled in the art can select appropriate amounts of an MT-containing compound to use in oral formulations.

As explained above, the MT weight factor for LMTM is 1.67. Since it is convenient to use unitary or simple fractional amounts of active ingredients, non-limiting example LMTM dosage units may include 17 mg etc.

In one embodiment there is provided a dosage unit pharmaceutical composition which comprises about 17, 27, 34, 51 mg etc. of LMTM.

Subjects, Patients and Patient Groups

In some embodiments the subject may be a human who has been diagnosed as having (“confirmed”) COVID-19, or wherein said method comprises making said diagnosis.

Diagnosis of COVID-19 may be via any method known in the art. Examples include laboratory testing for the presence of the SARS-CoV-2 virus—for example directly based on the presence of virus itself (e.g. using RT-PCR and isothermal nucleic acid amplification, or the presence of antigenic proteins) or indirectly via antibodies produced in response to infection. Other methods of diagnosis include chest X-ray, optionally in combination with characteristic symptoms as described below (see e.g. Li, Xiaowei, et al. “Molecular immune pathogenesis and diagnosis of COVID-19.” Journal of Pharmaceutical Analysis (2020); Fang, Yicheng, et al. “Sensitivity of chest CT for COVID-19: comparison to RT-PCR.” Radiology (2020): 200432; Chan, Jasper Fuk-Woo, et al. “Improved Molecular Diagnosis of COVID-19 by the Novel, Highly Sensitive and Specific COVID-19-RdRp/Hel Real-Time Reverse Transcription-PCR Assay Validated In Vitro and with Clinical Specimens.” Journal of Clinical Microbiology 58.5 (2020); Tang, Yi-Wei, et al. “The laboratory diagnosis of COVID-19 infection: current issues and challenges.” Journal of Clinical Microbiology (2020).

In some embodiments the subject is one:

(1) requiring medical care for COVID-19 with definite evidence of SARS-CoV-2 infection e.g. nucleic acid based diagnosis,

(2) having an SpO2 less than 95% on room air at screening, and

(3) having radiographic evidence of pulmonary infiltrates.

In some embodiment the subject is a human who has been assessed as being “at risk” of, COVID-19, or having probable COVID-19 e.g. based on situational or other data.

Those are particular risk of COVID-19 include:
People who have been in close contact with one or more COVID-19 cases
People 65 years and older;
People who live in a nursing home, care home, or long-term care facility;
People of all ages with relevant underlying medical conditions, particularly if not well controlled, including:
   People with chronic lung disease or moderate to severe asthma
   People who have serious heart conditions
   People who are immunocompromised
      As is known in the art, many conditions can cause a person to be immunocompromised, including cancer treatment, smoking, bone marrow or organ transplantation, immune deficiencies, poorly controlled HIV or AIDS, and prolonged use of corticosteroids and other immune weakening medications
   People with severe obesity (body mass index [BMI] of 40 or higher)
   People with diabetes
   People with chronic kidney disease undergoing dialysis
   People with liver disease

Symptoms or circumstances which are indicative of potential (“probable”) COVID-19 include:

1) a patient with acute respiratory tract infection (sudden onset of at least one of the following: cough, fever, shortness of breath) AND with no other aetiology that fully explains the clinical presentation AND with a history of travel or residence in a country/area reporting local or community transmission during the 14 days prior to symptom onset;

OR

2) a patient with any acute respiratory illness AND having been in close contact with a confirmed or probable COVID-19 case in the last 14 days prior to onset of symptoms;

OR

3) A patient with severe acute respiratory infection (SARI) (fever and at least one sign/symptom of respiratory disease (e.g., cough, fever, shortness breath)) AND requiring hospitalisation AND with no other aetiology that fully explains the clinical presentation. “Close contact” as used herein is defined as:
A person living in the same household as a COVID-19 case;
A person having had direct physical contact with a COVID-19 case (e.g. shaking hands);
A person having unprotected direct contact with infectious secretions of a COVID-19 case (e.g. being coughed on, touching used paper tissues with a bare hand);
A person having had face-to-face contact with a COVID-19 case within 2 metres and >15 minutes;
A person who was in a closed environment (e.g. classroom, meeting room, hospital waiting room, etc.) with a COVID-19 case for 15 minutes or more and at a distance of less than 2 metres;
A healthcare worker (HCW) or other person providing direct care for a COVID-19 case, or laboratory workers handling specimens from a COVID-19 case without recommended personal protective equipment (PPE) or with a possible breach of PPE;
A contact in an aircraft sitting within two seats (in any direction) of the COVID-19 case, travel companions or persons providing care, and crew members serving in the section of the aircraft where the index case was seated (if severity of symptoms or movement of the case indicate more extensive exposure, passengers seated in the entire section or all passengers on the aircraft may be considered close contacts).

The epidemiological link to a probable or confirmed case may have occurred within a 14-day period before the onset of illness in the suspected case under consideration. Given the overlap in the population characteristics between those at risk of AD and COVID-19 (for example care home populations), and the safety of LMTX in this at-risk population, the treatments of the present invention may in principle be performed in conjunction with treatments for the purpose of AD.

The patient may be an adult human, and the population-based dosages described herein are premised on that basis (typical weight 50 to 70 kg). If desired, corresponding dosages may be utilised for subjects falling outside of this range by using a subject weight factor whereby the subject weight is divided by 60 kg to provide the multiplicative factor for that individual subject.

Labels, Instructions and Kits of Parts

The unit dosage compositions described herein (MT-containing compound plus optionally other ingredients) may be provided in a labelled packet along with instructions for their use.

In one embodiment, the pack is a bottle, such as are well known in the pharmaceutical art. A typical bottle may be made from pharmacopoeial grade HDPE (High-Density Polyethylene) with a childproof, HDPE pushlock closure and contain silica gel desiccant, which is present in sachets or canisters. The bottle itself may comprise a label, and be packaged in a cardboard container with instructions for us and optionally a further copy of the label.

In one embodiment, the pack or packet is a blister pack (preferably one having aluminium cavity and aluminium foil) which is thus substantially moisture-impervious. In this case the pack may be packaged in a cardboard container with instructions for us and label on the container.

Said label or instructions may provide information regarding COVID-19 or SARS-CoV-2.

Methods of Treatment

Another aspect of the present invention, as explained above, pertains to a method of treatment of COVID-19 comprising administering to a patient in need of treatment a prophylactically or therapeutically effective amount of a compound as described herein, preferably in the form of a pharmaceutical composition.

Use in Methods of Therapy

Another aspect of the present invention pertains to a compound or composition as described herein, for use in a method of treatment of COVID-19 of the human or animal body by therapy.

Use in the Manufacture of Medicaments

Another aspect of the present invention pertains to use of an MT compound or composition as described herein, in the manufacture of a medicament for use in treatment of COVID-19.

In some embodiments, the medicament is a composition e.g a dose composition as described herein.

Mixtures of Oxidised and Reduced MT Compounds

The LMT-containing compounds utilised in the present invention may include oxidised (MT⁺) compounds as ‘impurities’ during synthesis, and may also oxidize (e.g., autoxidize) after synthesis to give the corresponding oxidized forms. Thus, it is likely, if not inevitable, that compositions comprising the compounds of the present invention will contain, as an impurity, at least some of the corresponding oxidized compound. For example an “LMT” salt may include up to 15% e.g. 10 to 15% of MT⁺ salt.

When using mixed MT compounds, the MT dose can be readily calculated using the molecular weight factors of the compounds present.

Salts and Solvates

Although the MT-containing compounds described herein are themselves salts, they may also be provided in the form of a mixed salt (i.e., the compound of the invention in combination with another salt). Such mixed salts are intended to be encompassed by the term “and pharmaceutically acceptable salts thereof”. Unless otherwise specified, a reference to a particular compound also includes salts thereof.

The compounds of the invention may also be provided in the form of a solvate or hydrate. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, a penta-hydrate etc. Unless otherwise specified, any reference to a compound also includes solvate and any hydrate forms thereof.

Naturally, solvates or hydrates of salts of the compounds are also encompassed by the present invention.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1A: virucidal activity of MTC against SARS-CoV2 in vitro in the dark using Vero-E6 kidney cells (date from Cagno et al 2020).

FIG. 1B: data from FIG. 1A presented in terms of IC50 for antiviral activity.

FIGS. 2A and 2B: computational chemistry modelling of the high affinity LMT/M⁺-heme interaction.

FIG. 3A: calculation of human oral doses of MT provided as LMTX required to achieve the tissue concentrations required for inhibition of SARS-CoV-2 toxicity based on 20:1 tissue: plasma ratio determined from study in minipigs.

FIG. 3B: calculation as per FIG. 3A based on 40:1 tissue: plasma ratio inferred from minipig and rat autoradiography data.

FIG. 3C: calculation as per FIG. 3A based on 10:1 tissue: plasma ratio based on assumed lower lung penetration.

FIG. 3D: calculation as per FIG. 3A based on 80:1 tissue: plasma ratio, for reference.

FIG. 4: calculations for IV dosing based on 20:1 tissue: plasma ratio. The dose required as continuous infusion (mg/hr) is shown in FIG. 4A and for bolus doses given 6-hourly is shown in FIG. 4B

FIG. 5: calculations for IV dosing based on 40:1 tissue: plasma ratio. FIGS. 5A and 5B provide the corresponding estimates for continuous infusion or infusion over 5 minutes every 6 hrs.

FIG. 6: calculations for IV dosing based on 10:1 tissue: plasma ratio. FIGS. 6A and 6B provide the corresponding estimates for continuous infusion or infusion over 5 minutes every 6 hrs.

FIG. 7: calculations for IV dosing based on 80:1 tissue: plasma ratio for reference. FIGS. 7A and 7B provide the corresponding estimates for continuous infusion or infusion over 5 minutes every 6 hrs.

FIG. 8: oxygen saturation levels in patients receiving LMTX compared pre-dose and after 4 hrs in the clinic following administration of a single doses of LMT at 4 mg and ˜100mg (mean of 75 mg, 100 mg, 125 mg). Levels were measured pre-dose and 4 hrs after dosing (post-dose).

FIG. 9: the effects of LMTM on SpO2 levels over 4 hours was independent of any corresponding effect on metHb.

FIG. 10: LMTM at high dosages over a period of time systematically increases metHb levels.

### REFERENCE EXAMPLE 1

### Methylthioninium Chloride (MTC) as an Antiviral

MTC (methylthioninium chloride, methylene blue) has been available as a drug since 1876. It is on the world health organisation's list of essential medicines, which is a list of the safest and most effective medicines in a health system.

MTC has been applied previously in many areas of clinical medicine including treatment of methemoglobinemia, malaria, nephrolithiasis, bipolar disorder, ifosfamide encephalopathy and most recently in Alzheimer disease (AD; Wischik et al., 2015; Nedu et al 2020).

Several studies have investigated the antiviral activity of MTC. One such study reported a significant reduction in viral load in hepatitis C patients at a dose of 130 mg/MTC per day (i.e. 98 mg/MT-equivalent per day) for 50 days (Wood et al., 2006; Mehta et al., 2006). Photoactivated MTC is routinely used for viral sterilisation of blood products in vitro via a photo-oxidation mechanism whereby intercalated methylthioninium (MT) generates singlet oxygen following photo-activation which damages and breaks nucleic acids and inactivates viruses. Viruses susceptible to MTC treatment include HIV-1 and 2, herpes and hepatitis C (Muller-Breitkreutz 1998, Mohr, 1999).

Recently, there is increasing interest in MTC as a potential treatment for COVID-19. MTC inhibits binding of coronavirus spike protein to its main receptor, angiotensin-converting enzyme 2 (ACE2) through which the virus gains entry into cells (IC₅₀ of 3.0 μM or 0.09 μg/ml; Bojadzic et al 2020).

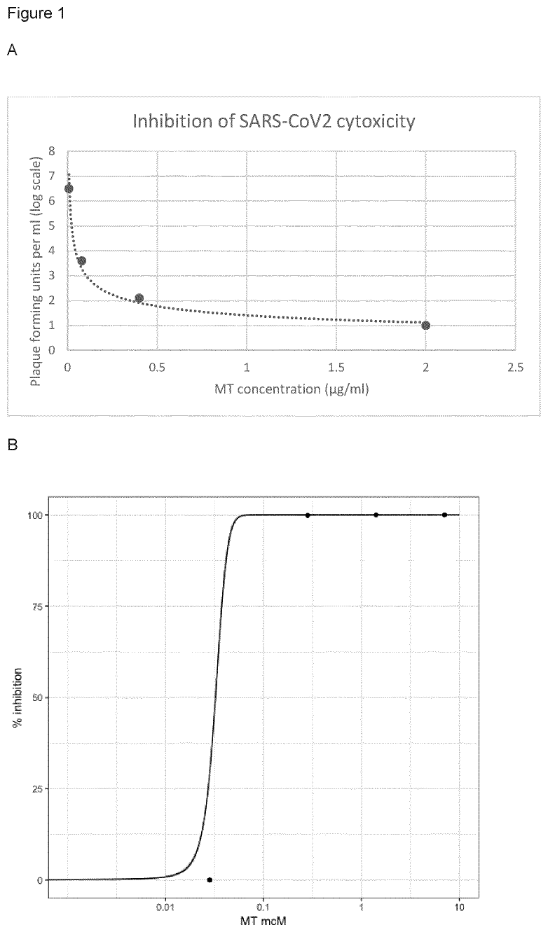

A recent study published by Cagno and colleagues reported that MTC had virucidal activity against SARS-CoV2 in vitro in the dark using Vero-E6 kidney cells (Cagno et al 2020; FIG. 1A). The data have been replotted as percentage inhibition of SARS-CoV-2 toxicity to permit estimation of the IC₅₀ for antiviral activity (FIG. 1B). The mechanism responsible for this viricidal effect is unknown, but, as explained further below, it is most likely mediated via the reduced form of the MT moiety (leuco-MT, LMT), since MT needs to be converted to the LMT form to gain entry into cells (Merker et al., 1997; May et al., 2004).

Using the Cagno et al. data, the calculated IC₅₀ of the MT moiety for neutralising viral toxicity in a Vero cell assay is 0.032 μM at 20 hrs. The IC₅₀ values for SARS-CoV-2 antiviral activity of two other compounds (hydroxychloroquine and remdesivir) in a similar Vero cell assay using viral replication as the endpoint has been reported (Yao et al., 2020; Wang et al., 2020). For hydroxychloroquine, the IC₅₀ values are 6.25 μM at 24 hrs and 0.72 μM at 48 hrs. For remdesivir, the IC₅₀ value is 0.77 μM at 48 hours. Therefore, assuming comparability of the assays, LMT appears to be approximately 23-fold more potent as a SARS-CoV-2 antiviral. The relatively low potency of hydroxychloroquine limits its clinical utility, since the upper limit of safe dosing is 400 mg/day, whereas the clinical dose required to achieve optimal antiviral activity is approximately 800 mg/day (Yao et al., 2020). Therefore, the typical dosing regimen for hydroxychloroquine is limited to 800 mg on day, followed by 400 mg per day on days 2-7.

### EXAMPLE 2

### Hydromethylthionine Salts as a Monotherapy for Covid 19

The MT moiety can exist in the oxidised MT⁺ form and in the reduced LMT form (Harrington et al., 2015;).

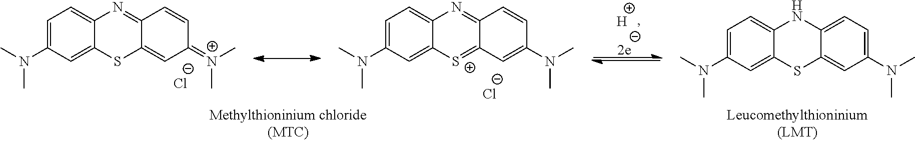

MTC is the chloride salt of the oxidised MT⁺ form. It needs to be converted to the reduced leuco-MT (LMT; international non-proprietary name: hydromethylthionine) form by a thiazine dye reductase activity in the gut to permit absorption and distribution to deep compartments including red cells and brain (Baddeley et al., 2015). Likewise, in isolated red cell preparations, MT⁺ needs to be converted to LMT to permit uptake both into red cells (May et al., 2004) and into pulmonary endothelial cells (Merker et al., 1997).

Because MTC is actually a prodrug for LMT, the predominant form in the body, TauRx developed a stabilised reduced form of MT as LMTM (leuco-methylthioninium bis(hydromethanesulphonate); hydromethylthionine mesylate) in order to permit direct administration of the LMT form.

Synthesis of LMTX and LMTM compounds can be performed according to the methods described in the art (see e.g. WO2007/110627, and WO2012/107706)

Mitochondrial Dysfunction in COVID-19

There is mounting evidence to suggest a link between COVID-19 and mitochondrial dysfunction (Saleh et al 2020; Singh et al 2020). A significant number of COVID-19 patients develop severe consequences attributed to a surge of inflammatory events described as the “cytokine storm”. Mitochondria play a pivotal role in maintaining cellular oxidative homeostasis and a heightened inflammatory response is thought to lead to mitochondrial dysfunction in these patients. Mitochondria are the main source of reactive oxygen species (ROS) within the cells. Increased ROS generation causes both intra- and extracellular mitochondrial damage which in turn leads to microbiota dysbiosis and platelet dysfunction which plays a major role in blood clotting and coagulopathy events that further aggravate the inflammatory response in a vicious cycle of events contributing to COVID-19 disease progression (Melchinger et al 2019). A recent study aiming to determine which parts of the human interactome are most affected by SARS-CoV-2-infection demonstrated that a member of the mitochondrial complex I is downregulated by infection leading to apoptosis and ultimately cell death (Guzzi et al 2020). In addition, Singh and colleagues (2020) reported that SARS-CoV-2 upregulated genes in the interferon, cytokines, nuclear factor kappa B (NF-κB) and ROS processes, while downregulating mitochondrial organisation and respiratory processes, in a lung cell line.

The above studies indicate that mitochondrial dysfunction may represent an important mediator in the development of COVID-19 and could contribute to the dysregulated immune response of COVID-19 patients, resulting in accelerated progression of the disease and a hyper-inflammatory state.

LMTM has been shown to enhance mitochondrial function both in vitro (Atamna & Kumar 2010) and in vivo (Riedel et al., 2020). This is due to the fact that MT⁺/LMT has a redox potential close to zero which is mid-way between the potentials of Complex I and Complex IV in the mitochondrial electron transport chain and can therefore act as an electron shuttle. This activity translates into an anti-ischaemic activity which limits the extent of infarction in a unilaterally ligated rat-brain model of cerebral ischaemia (Rodriguez et al., 2014). Therefore, LMT has the ability to protect tissues in the context of hypoxia where oxygen delivery is limiting.

In addition to enhancing mitochondrial function, MT dosed orally as MTC has been shown to increase mitochondrial biogenesis (Stack et al., 2014). Enhancement of mitochondrial biogenesis is linked to cellular clearance mechanisms, such as macroautophagy, pathways related to scavenging of ROS as well as the ability to increase in Nrf2 levels (Gureev et al., 2016). De la Vega and colleagues (2016) argue in an extensive review that Nrf2 plays an important protective role with respect to oxidative and inflammatory lung damage in Acute Lung Injury/Acute Respiratory Distress Syndrome (ADI/ARDS). They present evidence to show that pharmacological activation of Nrf2 would be expected to ameliorate alveolar damage from the primary infection but also from mechanical and hyperoxic injury resulting from Ventilation Induced Lung Injury (VILI). Oral dosing with MTC at 30 mg/kg has been shown to increase Nrf2 levels in brain (Stack et al., 2014). As noted above, the oxidised MT+ needs to be reduced to LMT to permit uptake into pulmonary endothelial cells (Merker et al., 1997). It is therefore credible that LMTM would have similar ability to induce Nrf2 in ADI/ARDS.

Blood Oxygen Carrying Capacity

COVID-19 has been associated with the emergence of both methemoglobinemia and hypoxaemia in patients (Naymagon et al., 2020). Methemoglobinemia results from oxidation of the iron contained in haemoglobin from the ferrous (Fe²⁺) to the ferric (Fe³⁺) form. The oxidation is associated with a decrement in the capacity of haemoglobin to carry oxygen efficiently (Curry et al., 1982). MTC is the primary treatment for methemoglobinemia, and indeed represents the only approved indication for its clinical use. The oxidised MT⁺ form of methylthionine given as MTC is first reduced to LMT at the cell surface as a prerequisite for red cell entry (May et al., 2004). It is then LMT which is the active species at the heme site, binding to porphyrin and permitting the transfer of an electron which converts Fe³⁺ to Fe²⁺, thereby restoring normal oxygen-carrying capacity (Yubisui et al., 1980; Blank et al., 2012).

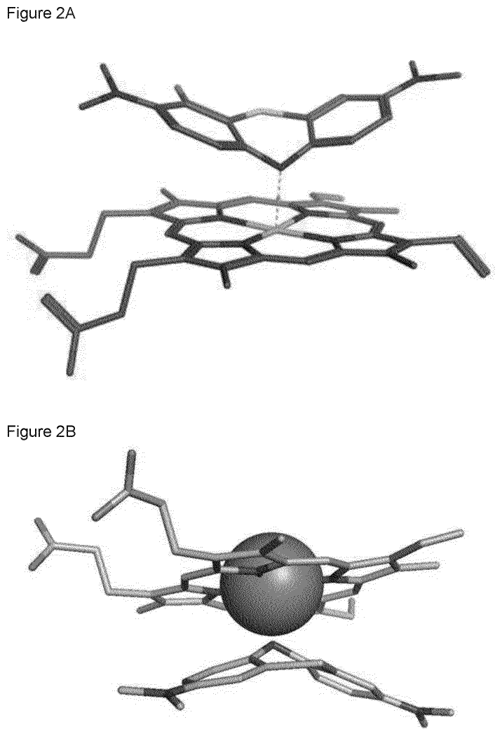

Computational chemistry modelling shown in FIGS. 2A and B provides a structural basis explaining the dynamics of the high affinity LMT-heme interaction. The LMT nitrogen coordinates with the heme iron atom by orientating itself towards the iron atom within 2.1 Å (dotted line in FIG. 2A). In methaemoglobinaemia, the iron atom is in the oxidised Fe³⁺ state.

In conditions associated with hypoxaemia where the iron the iron atom is in the Fe²⁺ state, the close formation of the LMT/heme coordinate facilitates oxygen carrying capacity via a process that does not require the transfer of an electron. When Hb is in the deoxygenated state, the heme is in the domed T state with Fe not fully accommodated in the tetrapyrrole ring, and is held by two histidines (His 87 in alpha subunit/His 92 in beta subunit and His 58 in alpha subunit/His 63 in beta submit). In this state, the ionic radius of the iron, which is in a high-spin Fe(II) state, is too large (radius 2.06 Å) to fit in the ring of nitrogens with which it coordinates; it is 0.6 Å out of the plane of the ring. When O₂ binds to the heme group it assumes the R state, becomes planar and the iron ion lies in the plane of the ring, as it is in a low-spin Fe(II) state with a smaller radius (1.98 Å). All six coordination positions of the ion are occupied: the bound oxygen molecule accounts for the sixth. When O₂ binds to Fe²⁺, it displaces the distal histidine and stabilises the heme moiety in the flat R-state. The binding of oxygen by haemoglobin is cooperative. As the haemoglobin tetramer units bind successive oxygens, the oxygen affinity of the subunits increases. The affinity for the fourth oxygen to bind is approximately 300 times that for the first. LMT is able to bind to the Fe of heme with an estimated field factor of 1.2-1.5. The field factor of LMT is sufficient to bind to Fe²⁺ (potentially f-factor of 1.2-1.5; C K Jorgensen, Oxidation numbers and oxidation states, Springer 1969 pp84-30 85). MT is therefore a strong field ligand and is able to bind to heme sufficiently to induce an R-state configuration within the protein. The LMT moiety is able to form a complex with Fe²⁺ by donation of lone pair electrons from the N atom to the d-orbitals of ferrous iron (Molecules 2013, 18(3), 3168-3182; https://doi.org/10.3390/molecules18033168). Therefore, binding of LMT overcomes the initial energy barrier for oxygen binding, which is thereafter able to bind and oxygenate all four heme groups of haemoglobin. Because O₂ binds with higher affinity, it is able to displace LMT from the same binding site. This permits normal oxygen dissociation to occur with release of bound oxygen to peripheral tissues at low pH/high pCO₂.

Given that the LMT is the active form, the clinical evidence below showing that LMTM treatment enhances the oxygen carrying capacity of the blood confirms that this LMT-heme interaction facilitates oxygen uptake by haemoglobin.

Potential for LMTM to Improve CNS Sequelae of COVID-19

There are emerging clinical reports indicate that COVID-19 may have detrimental effects on the central nervous system (De Felice et al 2020; Baig et al 2020). It has been reported that SARS-CoV-2 preferentially targets soma of cortical neurons but not neural stem cells, the target cell type of ZIKA virus (Ramani et al 2020). Imaging analysis also revealed that SARS-CoV-2 co-localises with tau is associated with missorting tau and subsequent neuronal death.

LMTM was originally developed as a treatment for pathological tau protein aggregation in AD and other dementias. Therefore, LMTM may have a role to play in limiting the long-term functional disability and cognitive impairment that has been reported in some cases of COVID-19 infection (Zhou et al., 2020).

### EXAMPLE 3

### Estimation of Clinical Dose of LMTM Required for SAR-CoV-2 Antiviral Activity

TauRx originally focused on MTC as a potential treatment for AD because of its ability to block pathological aggregation of the microtubule associated protein tau which forms neurofibrillary tangles and is responsible for clinical dementia in Alzheimer's Disease (Wischik et al., 1996; Harrington et al., 2015).

Comparatively, LMTM shows better pharmacodynamic and pharmacokinetic properties than MTC (Harrington et al., 2015; Baddeley et al., 2015). Following oral administration, free plasma MT/LMT is subject to efficient first-pass metabolism which converts it to an inactive conjugate, and which is the predominant species in found plasma. The 20-fold better uptake into red cells is important for protection LMT from metabolic inactivation and permitting its efficient distribution to the brain and other tissue compartments (Baddeley et al., 2015).

An initial Phase 2 dose-finding study identified 138 mg/day as the minimum effective dose of MTC (Wischik et al., 2015). However, because LMT absorption from LMTM is much more efficient, the minimum effective dose required for anti-dementia effects was found to be 8 mg/day, and 16 mg/day was found to be the optimally effective dose (Schelter et al., 2019).

The reason for this has been elucidated in two unpublished preclinical studies which provide highly relevant insights into the use of LMTX for treating COVID-19:

A pharmacokinetic study in minipigs (nearest to humans in terms of pharmacokinetic properties) given LMTM orally at doses corresponding to human doses of 8, 24, 40, 71 and 155 mg/day found that the mean brain:plasma ratio at 2 and 4 hrs for the parent LMT moiety is ˜20:1 (compared to 0.3:1 for MTC).

A further whole body autoradiography study rats compared the distribution of LMT-associated radioactivity in brain, lung and heart following oral dosing at 10 mg/kg. This found that the ratio of heart and lung to brain is 2:1. However, this is for total MT, including the inactive conjugate. The ratio specific for LMT in lung is therefore unknown. It is possible to relate plasma levels determined in a large clinical population (Schelter et al., 2019) to expected tissue levels of LMT at steady state across a wide dosing range of 8-250 mg/day. However, this depends critically on the tissue:plasma ratio for specifically affected tissues such a lung.

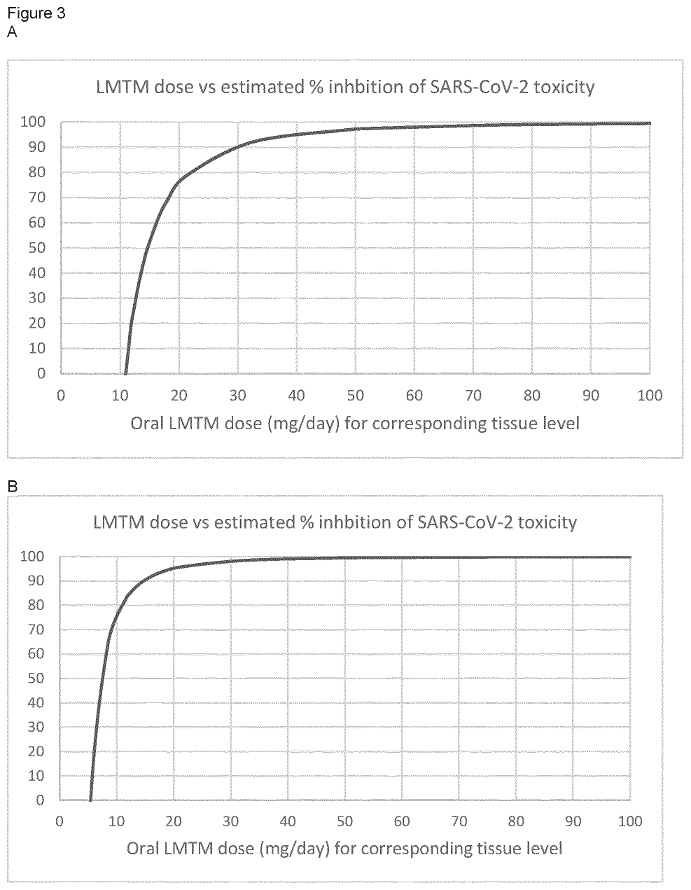

Combining the human and animal PK data, it is possible to calculate the human doses required to achieve the tissue concentrations required for inhibition of SARS-CoV-2 toxicity as reported by Cagno et al. (2020). This is shown in FIG. 3A below using the 20:1 tissue: plasma ratio determined from study in minipigs. The dose required to achieve 99% reduction in toxicity in 95% population would be approximately 60 or 75 mg/day.

However, other scenarios should also be considered. If the tissue:plasma ratio is 40:1 (consistent with the minipig and rat autoradiography data), a dose of approximately 40 mg/day would be sufficient (FIG. 3B).

Furthermore if the plasma:lung ratio is closer to 10:1, then the dose required for 99% reduction in toxicity would be closer to 150 mg/da (FIG. 3C).

For reference FIG. 3D illustrates the corresponding estimates for the tissue:plasma ratio of 80:1. The dose required to achieve at least 99% inhibition of toxicity in at least 95% of population is approximately 20 mg/day.

Therefore, until further tissue-specific data are available for the tissue distribution of LMT (as distinct from total MT) in lung in particular, an appropriate dosing range would be at least 30 mg/day.

The safety of LMTM across doses ranging from 8-250 mg/day has been well established from three Phase 3 trials in over 2,000 patients with dementia. Therefore, doses up to 250 mg/day could be given safely for treatment of COVID-19 patients.

IV Dosing

The predicted tissue levels at IV doses depend on the bioavailability and the tissue:plasma ratio (study discussed above).

We investigated bioavailability (oral vs iv) of LMTM in minipigs, based on total radioactivity following dosing of ¹⁴C-LMTM following dosing at 10 mg/kg oral and 5 mg/kg IV. Although absolute bioavailability adjusted for dose in this study was found to be ˜100%, we have assumed bioavailability of 75% for the purposes of dosage calculations.

For the reasons given above, a range of dosing regimes has been provided based on tissue:plasma ratios of 10:1, 20:1, 40:1, and 80:1 for reference.

The IV doses have been calculated for continuous infusion (mg/hr) or for IV bolus infusion administered 6-hourly. In each case, the infusion rates calculated from the population-PK model have been determined on the basis of the dose required for 95% of the population to have tissue levels above a given threshold required to achieve a given reduction in predicted SARS-CoV-2 tissue toxicity determined from the studies reported for Vero-E6 kidney cells for the MT moiety by Cagno et al. (2020).

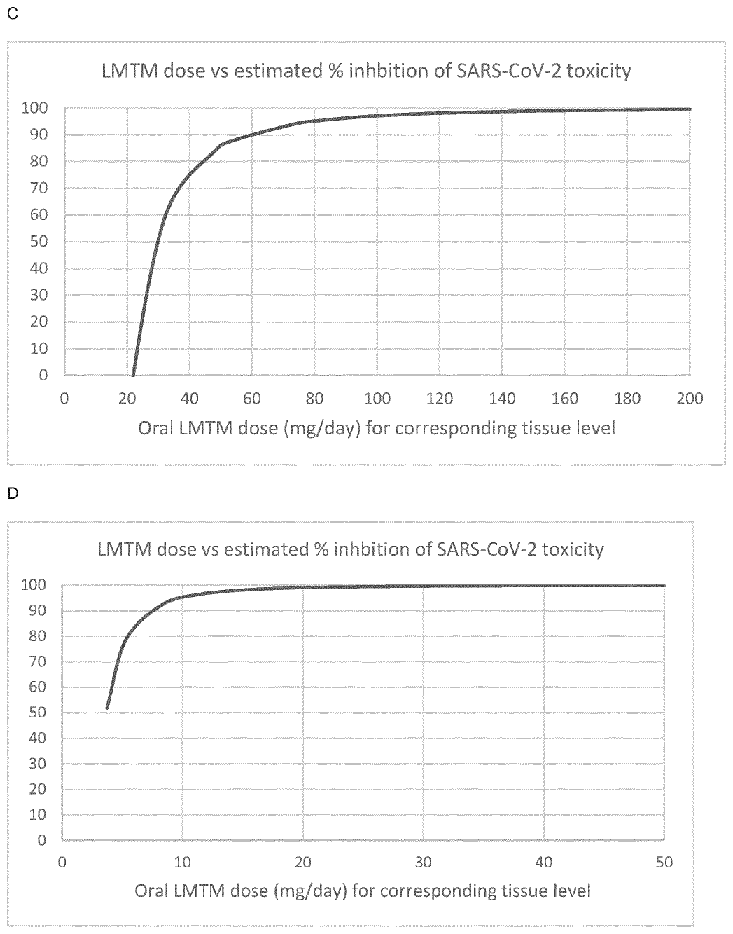

For the 20:1 tissue:plasma ratio, the dose required as continuous infusion (mg/hr) is shown in FIG. 4A and for bolus doses given 6-hourly is shown in FIG. 4B. The doses required to achieve at least 99% inhibition of toxicity in at least 95% of the population are 2.8 mg/hr as continuous infusion or 20 mg as infusion over 5 minutes every 6 hrs or 27 mg as infusion over 5 minutes every 8 hrs, or 40 mg as infusion over 5 minutes every 12 hrs,

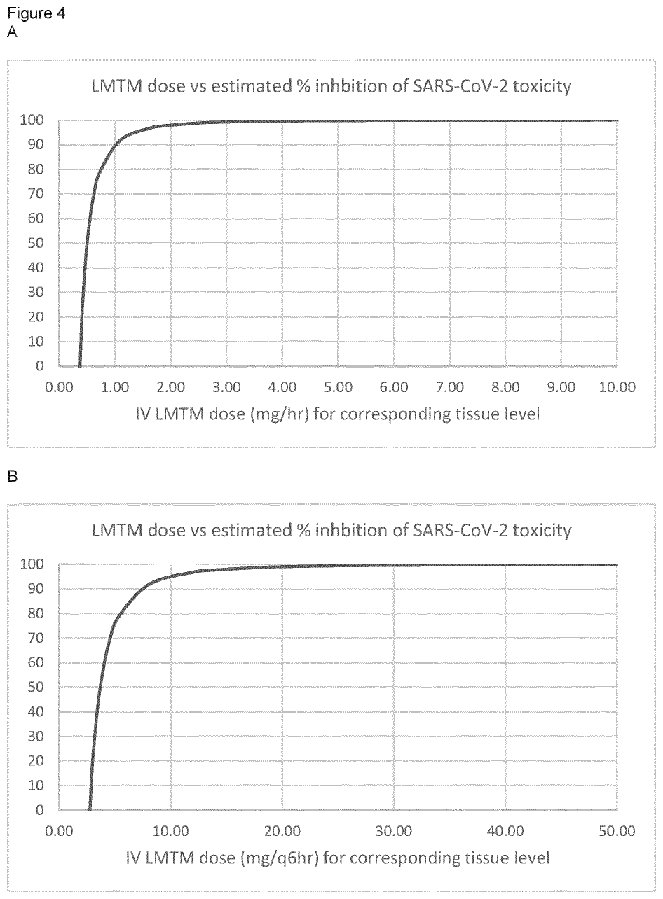

FIGS. 5A and 5B provide the corresponding estimates for the tissue:plasma ratio of 40:1. The doses required to achieve at least 99% inhibition of toxicity in at least 95% of the population are 1.2 mg/hr as continuous infusion or 12 mg as infusion over 5 minutes every 6 hrs or 16 mg as infusion over 5 minutes every 8 hrs, or 24 mg as infusion over 5 minutes every 12 hrs,

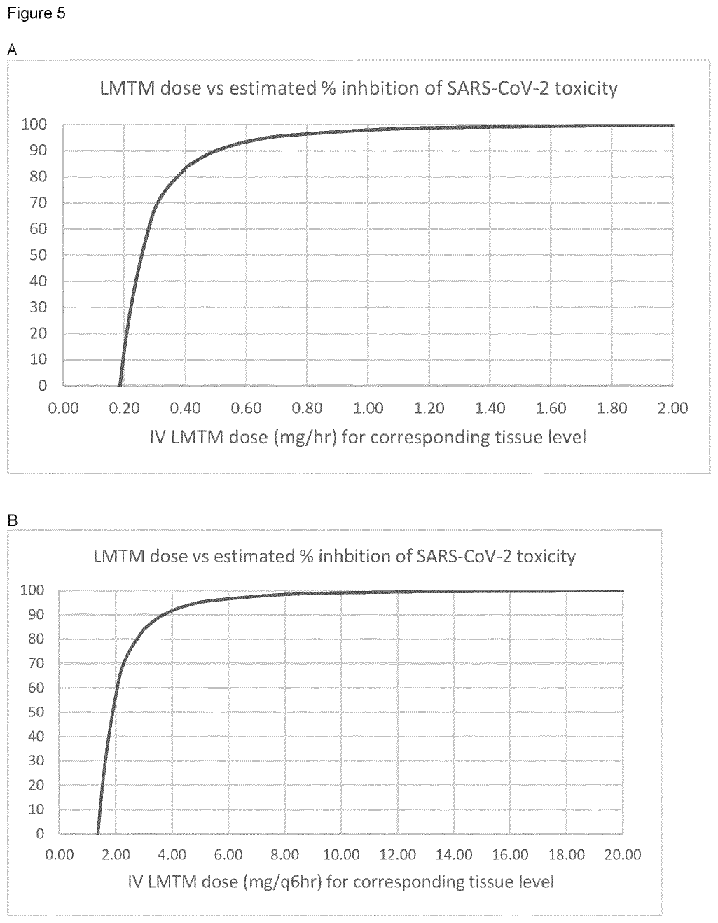

FIGS. 6A and 6B provide the corresponding estimates for the tissue:plasma ratio of 10:1. The doses required to achieve at least 99% inhibition of toxicity in at least 95% of the population are 6.8 mg/hr as continuous infusion or 50 mg as infusion over 5 minutes every 6 hrs or 67 mg as infusion over 5 minutes every 8 hrs, or 100 mg as infusion over 5 minutes every 12 hrs,

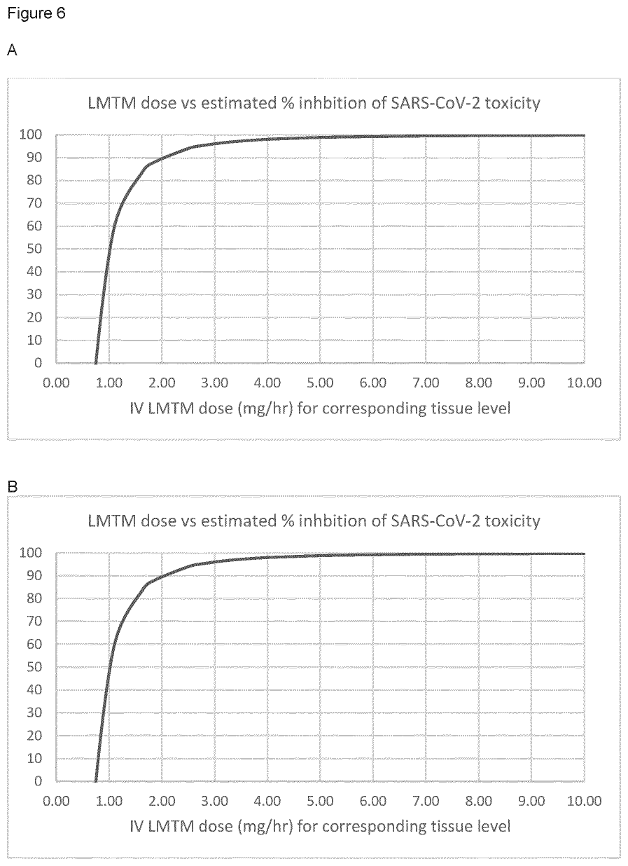

For reference, FIGS. 7A and 7B provide the corresponding estimates for the tissue:plasma ratio of 80:1. The doses required to achieve at least 99% inhibition of toxicity in at least 95% of the population are 0.7 mg/hr as continuous infusion or 5.3 mg as infusion over 5 minutes every 6 hrs or 7 mg as infusion over 5 minutes every 8 hrs, or 21 mg as infusion over 5 minutes every 12 hrs,

### EXAMPLE 4

### Preliminary Clinical Data

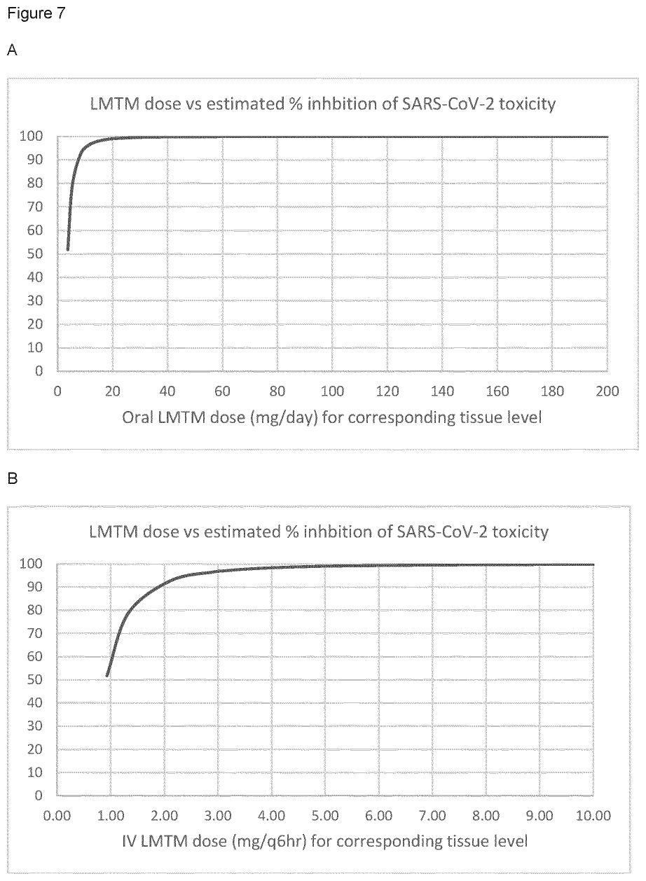

The present inventors have used data available for patients participating in clinical trials to determine whether LMT enhances oxygen saturation of blood. Data were available for 18 subjects with oxygen saturation <94% at baseline (lower limit of normal range is 95%). Oxygen saturation levels were compared pre-dose and after 4 hrs in the clinic following administration of a single doses of LMT at 4 mg and ˜100mg (mean of 75, 100, 125 mg; FIG. 8).

LMTM at both dosing ranges significantly increased oxygen saturation at 4 hours, again supporting multiple beneficial modes of action for LTMX for treatment of COVID-19 patients.

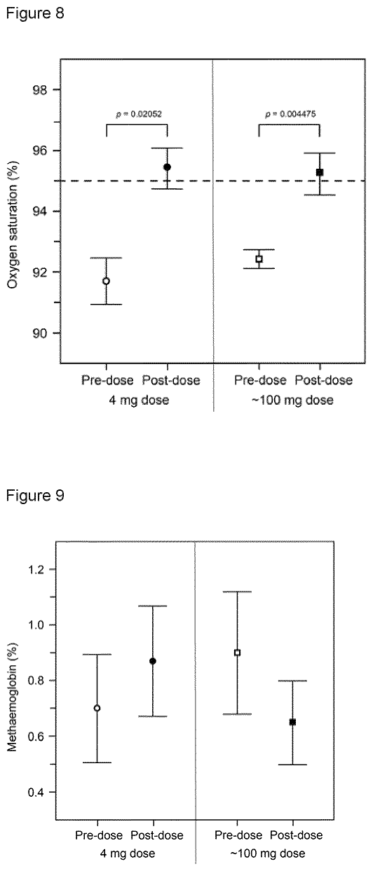

In order to understand this effect further the inventors investigated whether the low oxygen saturation in these patients is due to elevation in metHb levels. There was no difference in metHb levels at baseline between subjects with low SpO2 and those with SpO2 levels in the normal range. Furthermore, the effects on LMTM on SpO2 levels over 4 hours was independent of any corresponding effect on metHb (FIG. 9).

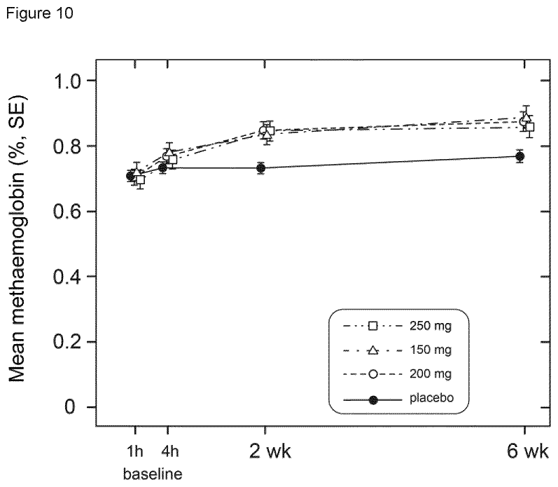

Therefore, LMTM is able to act on oxygen saturation in the blood by a novel mechanism unrelated to its known effects on metHb. Indeed LMTM at higher doses systematically increases metHb levels (FIG. 10).

### EXAMPLE 5

### A Clinical Trial for LMTM for Treatment of Covid-19

LMTM may be given at doses of 60 and 120 mg/day, or alternatively 75mg/day or 150mg/day (see Example 3 above), over 1 month to adult patients who are currently hospitalised and requiring medical care for COVID-19 with definite evidence of SARS-CoV-2 infection from nasal swab, who have an SpO2 less than 95% on room air at screening or PaO2/FiO2 <300 or respiratory rate≥20 per minute and have radiographic evidence of pulmonary infiltrates.

Patients already participating in any other clinical trial of an experimental agent treatment for COVI D-19, or in whom concurrent treatment or planned concurrent treatment with other agents with actual or possible direct acting antiviral activity against SARS-CoV-2, or who require mechanical ventilation at screening may be excluded, as will patients with a calculated creatinine clearance<30 ml/min.

The principal endpoints are change in clinical disease severity (7-point ordinal scale; Table 1), SpO2 change measured by Co-Oximeter, change in viral burden measured by PCR of nasal swabs, C-reactive protein levels in blood, percentage of lung involvement on lung CT scan and mortality.

TABLE 1
7-point ordinal scale

1: Not hospitalised with no limitations on activities
2: Not hospitalised but with limitations on activities
3: Hospitalised, not receiving supplemental oxygen
4: Hospitalised, receiving supplemental oxygen
5: Hospitalised, receiving non-invasive ventilation
or high-flow nasal cannula
6: Hospitalised, receiving mechanical ventilation
7: Death

Based on publicly available data regarding the standard deviations on the key outcome measures, the number of subjects will be in the range of approximately 100 per arm.

### EXAMPLE 6

### Conclusion: Hydromethylthionine Salts as Treatment for COVID-19

For the foregoing rationale the LMTX class of compounds may provide benefits in the treatment (including prophylactic treatment) of COVID-19 patients both alone and in combination with other agents by reducing reducing viral toxicity at doses defined herein based on proprietary PK studies of LMTX in vivo. Also described herein are beneficial effects on blood increased oxygen saturation.

LMTX may also provide benefits to subjects in enhancing, mitochondrial function and improving CNS sequelae of COVID-19.

Furthermore, the LMTM does not have the cardiotoxicity that limits the dose and duration of certain other treatments.

### REFERENCES

Atamna, H., & Kumar, R. (2010). Protective role of methylene blue in Alzheimer's disease via mitochondria and cytochrome c oxidase. In Journal of Alzheimer's Disease (Vol. 20, Issue SUPPL.2). https://doi.org/10.3233/JAD-2010-100414
Baddeley, T. C., McCaffrey, J., M. D. Storey, J., Cheung, J. K. S., Melis, V., Horsley, D., Harrington, C. R., & Wischik, C. M. (2015). Complex Disposition of Methylthioninium Redox Forms Determines Efficacy in Tau Aggregation Inhibitor Therapy for Alzheimer's Disease. Journal of Pharmacology and Experimental Therapeutics, 352(1), 110-118. https://doi.org/10.1124/jpet.114.219352
Baig, A. M., Khaleeq, A., Ali, U., & Syeda, H. (2020). Evidence of the COVID-19 Virus Targeting the CNS: Tissue Distribution, Host-Virus Interaction, and Proposed Neurotropic Mechanisms. In ACS Chemical Neuroscience (Vol. 11, Issue 7, pp. 995-998). https://doi.org/10.1021/acschemneuro.0c00122
Blank, O., Davioud-Charvet, E., & Elhabiri, M. (2012). Interactions of the Antimalarial Drug Methylene Blue with Methemoglobin and Heme Targets in Plasmodium falciparum : A Physico-Biochemical Study. Antioxidants & Redox Signaling, 17(4), 544-554. https://doi.org/10.1089/ars.2011.4239
Bojadzic, D., Alcazar, O., & Buchwald, P. (2020). Methylene Blue Inhibits In Vitro the SARS-CoV-2 Spike-ACE2 Protein-Protein Interaction-A Mechanism That Can Contribute to Its Antiviral Activity Against COVID-19. BioRxiv, 2020.08.29.273441. https://doi.org/10.1101/2020.08.29.273441
Cagno, V., Medaglia, C., Cerny, A., Cerny, T., & Cerny, E. (2020). Methylene Blue has a potent antiviral activity against SARS-CoV-2 in the absence of UV-activation in vitro. BioRxiv, 2020.08.14.251090. https://doi.org/10.1101/2020.08.14.251090
De Felice, F. G., Tovar-Moll, F., Moll, J., Munoz, D. P., & Ferreira, S. T. (2020). Severe Acute Respiratory Syndrome Coronavirus 2 (SARS-CoV-2) and the Central Nervous System. In Trends in Neurosciences (Vol. 43, Issue 6, pp. 355-357). https://doi.org/10.1016/j.tins.2020.04.004
de la Vega, M. R., Dodson, M., Gross, C., Mansour, H. M., Lantz, R. C., Chapman, E., Wang, T., Black, S. M., Garcia, J. G. N., & Zhang, D. D. (2016). Role of Nrf2 and Autophagy in Acute Lung Injury. In Current Pharmacology Reports (Vol. 2, Issue 2, pp. 91-101). https://doi.org/10.1007/s40495-016-0053-2
Gureev, A. P., Syromyatnikov, M. Y., Gorbacheva, T. M., Starkov, A. A., & Popov, V. N. (2016). Methylene blue improves sensorimotor phenotype and decreases anxiety in parallel with activating brain mitochondria biogenesis in mid-age mice. Neuroscience Research, 113, 19-27. https://doi.org/10.1016/j.neures.2016.07.006
Guzzi, P. H., Mercatelli, D., Ceraolo, C., & Giorgi, F. M. (2020). Master Regulator Analysis of the SARS-CoV-2/Human Interactome. Journal of Clinical Medicine, 9(4), 982. https://doi.org/10.3390/jcm9040982
Harrington, C. R., Storey, J. M. D., Clunas, S., Harrington, K. A., Horsley, D., Ishaq, A., Kemp, S. J., Larch, C. P., Marshall, C., Nicoll, S. L., Rickard, J. E., Simpson, M., Sinclair, J. P., Storey, L. J., & Wischik, C. M. (2015). Cellular Models of Aggregation-dependent Template-directed Proteolysis to Characterize Tau Aggregation Inhibitors for Treatment of Alzheimer Disease. Journal of Biological Chemistry, 290(17), 10862-10875. https://doi.org/10.1074/jbc.M114.616029
May, J. M., Qu, Z. C., & Cobb, C. E. (2004). Reduction and uptake of methylene blue by human erythrocytes. American Journal of Physiology—Cell Physiology, 286(6 55-6). https://doi.org/10.1152/ajpce11.00512.2003
Mehta G, Mawdsley A et al., the effect of oral methylene blue on viral load in chronic hepatitis C infection. Poster presented at British association for the study of the liver (BASL) meeting. 2006 Sept. Dublin, Ireland.
Melchinger, H., Jain, K., Tyagi, T., & Hwa, J. (2019). Role of Platelet Mitochondria: Life in a Nucleus-Free Zone. Frontiers in Cardiovascular Medicine, 6. https://doi.org/10.3389/fcvm.2019.00153
Merker, M. P., Bongard, R. D., Linehan, J. H., Okamoto, Y., Vyprachticky, D., Brantmeier, B. M., Roerig, D. L., & Dawson, C. A. (1997). Pulmonary endothelial thiazine uptake: Separation of cell surface reduction from intracellular reoxidation. American Journal of Physiology—Lung Cellular and Molecular Physiology, 272(4 16-4). https://doi.org/10.1152/ajplung.1997.272.4.1673
Mohr, H., Bachmann, B., Klein-Struckmeier, A., & Lambrecht, B. (1997). Virus inactivation of blood products by phenothiazine dyes and light. Photochemistry and Photobiology, 65(3), 441-445. https://doi.org/10.1111//j1751-1097.1997.tb08586.x
Müller-Breitkreutz, K., & Mohr, H. (1998). Hepatitis C and human immunodeficiency virus RNA degradation by methylene blue/light treatment of human plasma. Journal of Medical Virology, 56(3), 239-245. https://doi.org/10.1002/(SICI)1096-9071(199811)56:3<239::A/D-JMV11>3.0.CO;2-9
Naymagon, L., Berwick, S., Kessler, A., Lancman, G., Gidwani, U., & Troy, K. (2020). The emergence of methemoglobinemia amidst the COVID-19 pandemic. Am J Hematol, 95, E196-E19. https://doi.org/10.1002/ajh.25868
Nedu, M. E., Tertis, M., Cristea, C., & Georgescu, A. V. (2020). Comparative study regarding the properties of methylene blue and proflavine and their optimal concentrations for in vitro and in vivo applications. In Diagnostics (Vol. 10, Issue 4). https://doi.org/10.3390/diagnostics10040223
Ramani, A., Müller, L., Ostermann, P. N., Gabriel, E., Abida-Islam, P., Müller-Schiffmann, A., Mariappan, A., Goureau, 0., Gruell, H., Walker, A., Andrée, M., Hauka, S., Houwaart, T., Dilthey, A., Wohlgemuth, K., Omran, H., Klein, F., Wieczorek, D., Adams, O., . . . Gopalakrishnan, J. (2020). SARS-CoV-2 targets cortical neurons of 3D human brain organoids and shows neurodegeneration-like effects. BioRxiv (Preprint), 2020.05.20.106575. https://doi.org/10.1101/2020.05.20.106575
Riedel, G., Klein, J., Niewiadomska, G., Kondak, C., Schwab, K., Lauer, D., Magbagbeolu, M., Steczkowska, M., Zadrozny, M., Wydrych, M., Cranston, A., Melis, V., Santos, R. X., Theuring, F., Harrington, C. R., & Wischik, C. M. (2020). Mechanisms of Anticholinesterase Interference with Tau Aggregation Inhibitor Activity in a Tau-Transgenic Mouse Model. Current Alzheimer Research, 17(3), 285-296. https://doi.org/10.2174/1567205017666200224120926
Rodriguez, P., Jiang, Z., Huang, S., Shen, Q., & Duong, T. Q. (2014). Methylene blue treatment delays progression of perfusion-diffusion mismatch to infarct in permanent ischemic stroke. Brain Research, 1588, 144-149. https://doi.org/10.1016/j.brainres.2014.09.007
Saleh, J., Peyssonnaux, C., Singh, K. C., & Edeas, M. (2020). Mitochondria and microbiota dysfunction in COVID-19 pathogenesis. Mitochondrion, 54, 1-7. https://doi.org/10.1016/j.mito.2020.06.008
Schelter, B. O., Shiells, H., Baddeley, T. C., Rubino, C. M., Ganesan, H., Hammel, J., Vuksanovic, V., Staff, R. T., Murray, A. D., Bracoud, L., Riedel, G., Gauthier, S., Jia, J., Bentham, P., Kook, K., Storey, J. M. D., Harrington, C. R., & Wischik, C. M. (2019). Concentration-Dependent Activity of Hydromethylthionine on Cognitive Decline and Brain Atrophy in Mild to Moderate Alzheimer's Disease. Journal of Alzheimer's Disease, 72(3), 931-946. https://doi.org/10.3233/JAD-190772
Singh, K. K., Chaubey, G., Chen, J. Y., & Suravajhala, P. (2020). Decoding sars-cov-2 hijacking of host mitochondria in covid-19 pathogenesis. In American Journal of Physiology—Cell Physiology (Vol. 319, Issue 2, pp. C258—C267). https://doi.org/10.1152/ajpce11.00224.2020
Stack, C., Jainuddin, S., Elipenahli, C., Gerges, M., Starkova, N., Starkov, A. A., Jove, M., Portero-Otin, M., Launay, N., Pujol, A., Kaidery, N. A., Thomas, B., Tampellini, D., Flint Beal, M., & Dumont, M. (2014). Methylene blue upregulates Nrf2/ARE genes and prevents tau-related neurotoxicity. Human Molecular Genetics, 23(14), 3716-3732. https://doi.org/10.1093/hmg/ddu080
Wang, M., Cao, R., Zhang, L., Yang, X., Liu, J., Xu, M., Shi, Z., Hu, Z., Zhong, W., & Xiao, G. (2020). Remdesivir and chloroquine effectively inhibit the recently emerged novel coronavirus (2019-nCoV) in vitro. Cell Res, 30(3), 269-271. https://doi.org/10.1038/s41422-020-0282-0
Wischik, C. M., Edwards, P. C., Lai, R. Y. K., Roth, M., & Harrington, C. R. (1996). Selective inhibition of Alzheimer disease-like tau aggregation by phenothiazines. Proceedings of the National Academy of Sciences, 93(20), 11213-11218. https://doi.org/10.1073/pnas.93.20.11213
Wischik, C. M., Staff, R. T., Wischik, D. J., Bentham, P., Murray, A. D., Storey, J. M. D., Kook, K. A., & Harrington, C. R. (2015). Tau Aggregation Inhibitor Therapy: An Exploratory Phase 2 Study in Mild or Moderate Alzheimer's Disease. Journal of Alzheimer's Disease, 44(2), 705-720. https://doi.org/10.3233/JAD-142874
Wood C, Nagy H. Methylene blue therapy of viral disease. US20060264423 A1, United States Patent and Trademark Office, 19 May 2006.
Yao, X., Ye, F., Zhang, M., Cui, C., Huang, B., Niu, P., Liu, X., Zhao, L., Dong, E., Song, C., Zhan, S., Lu, R., Li, H., Tan, W & Liu, D. (2020). In Vitro Antiviral Activity and Projection of Optimized Dosing Design of Hydroxychloroquine for the Treatment of Severe Acute Respiratory Syndrome Coronavirus 2 (SARS-CoV-2). Clin Infect Dis, 71(15), 732-739. https://doi.org/10.1093/cid/ciaa237
Zhou, H., Lu, S., Chen, J., Wei, N., Wang, D., Lyu,H., Shi, C., & Hua, S. (2020). The landscape of cognitive function in recovered COVID-19 patients. J Psychiatr Res. 129, 98-102. https://doi.org/10.1016/j.jpsychires.2020.06.022

1. A method of therapeutic treatment of COVID-19 in a subject,
which method comprises administering to said subject a methylthioninium (MT)-containing compound,
wherein said administration provides a total daily oral dose of between more than 30 to 250 mg of MT to the subject per day, optionally split into 2 or more doses,
or wherein said administration provides a total daily intravenous (IV) dose of between 10 and 200 mg of MT to the subject per day,
wherein the MT-containing compound is an LMTX compound of the following formula: 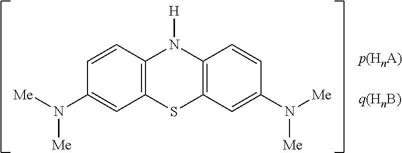
wherein each of HₙA and HₙB (where present) are protic acids which may be the same or different,
and wherein p=1 or 2; q=0 or 1; n=1 or 2; (p+q)×n=2,
or a hydrate or solvate thereof.

2. A method as claimed in claim 1 wherein the subject is a human who has been diagnosed with COVID-19, or wherein said method comprises making said diagnosis. 3 A method as claimed in claim 2 wherein the subject is characterised by having definite evidence of SARS-CoV-2 infection plus one, two or all of:
(1) requiring medical care for COVID-19;
(2) having an SpO2 less than 95% on room air;
(3) having radiographic evidence of pulmonary infiltrates.

4. A method of prophylactic treatment of COVID-19 in a subject,
which method comprises administering to said subject a methylthioninium (MT)-containing compound,
wherein said administration provides a total daily oral dose of between more than 30 to 250mg of MT to the subject per day, optionally split into 2 or more doses,
or wherein said administration provides a total daily intravenous (IV) dose of between 10 and 200 mg of MT to the subject per day,
wherein the MT-containing compound is an LMTX compound of the following formula: 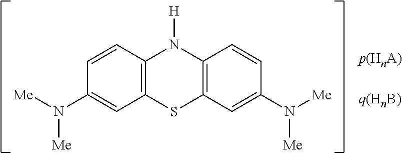
wherein each of HₙA and HₙB (where present) are protic acids which may be the same or different,
and wherein p=1 or 2; q=0 or 1; n=1 or 2; (p+q)×n=2,
or a hydrate or solvate thereof.

5. A method as claimed in claim 4 wherein the subject is a human who has been assessed as having suspected or probable COVID-19, and is optionally selected from: a subject who has been in close contact with one or more COVI D-19 cases; a subject who is at least 65 years old; a subject living in a nursing home, care home, or long-term care facility; a subject with an underlying medical condition which increases the likelihood of adverse effects from COVID-19.

6. A method as claimed in any one of claims 1 to 5 wherein the total daily oral dose is:
(i) greater than 35, 40, 50, or 60 mg and less than or equal to 250 mg of MT to the subject per day; and/or
(ii) greater than or equal to about 30.5, 30.6, 31, 35, 37.5, 40, 45, 50, 55, 60, 65, 70, 75, 80, 85, 90, 95, 100, 105, 110, 115, 120, 125, 130, 140, 150, 160, 170, 180, 190 or 200 mg MT.

7. A method as claimed in any one of claims 1 to 6 wherein the total daily oral dose is about 60, 75, 120, or 150 mg MT, which is optionally split twice a day or three times a day.

8. A method as claimed in any one of claims 1 to 5 wherein the total daily IV dose is between 26 and 200 mg of MT to the subject per day.

9. A method as claimed in any one of claims 1 to 5 wherein the total daily IV dose is:
(i) about 10, 11, 12, 13, 14, 15, 20, 25, 30, 35, 40, 45, 50, 60, 70, 80, 90, 100, 110, 120, 130, 140, 150, 160, 170, 180, 190 or 200 mg/day by continuous dosing.
(ii) about, 20, 25, 30, 35, 40, 45, 50, 60, 70, 80, 90, 100, 110, 120, 130, 140, 150 mg/day by bolus dosing.

10. A method as claimed in any one of claims 1 to 9 wherein the treatment is combined with a second agent.

11. A method as claimed in claim 10 wherein the second agent is selected from: chloroquine or hydroxychloroquine; lopinavir-ritonavir; arbidol; azithromycin, remdesivir, favipiravir, actemra; dexamethasone; convalescent plasma; a SARS-CoV-2-neutralizing antibody.

12. A method as claimed in claim 10 or claim 11 wherein the MT-containing compound and the second agent are administered sequentially within 12 hours of each other.

13. A method as claimed in any one of claims 10 to 12 wherein the subject is pre-treated with the second agent prior to commencement of the treatment with the MT-containing compound.

14. A method as claimed in claim 10 or claim 11 wherein the MT-containing compound and the second agent are administered simultaneously, optionally within a single dosage unit.

15. A method as claimed in any one of claims 1 to 14 wherein the MT-containing compound has the following formula, where HA and HB are different mono-protic acids:

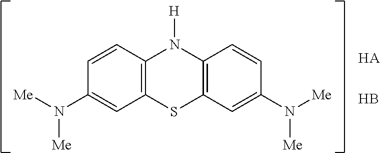

16. A method as claimed in any one of claims 1 to 14 wherein the MT-containing compound has the following formula: 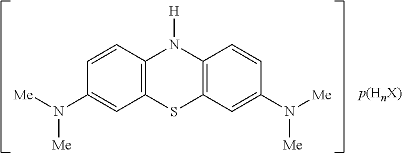
wherein each of HₙX is a protic acid.

17. A method as claimed in any one of claims 1 to 14 wherein the MT-containing compound has the following formula and H₂A is a di-protic acid:

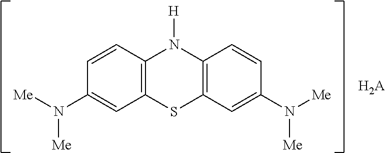

18. A method as claimed in claim 16 wherein the MT-containing compound has the following formula and is a bis-monoprotic acid:

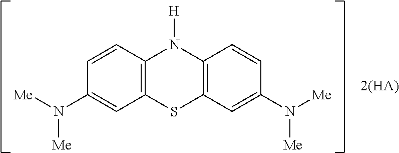

19. A method as claimed in any one of claims 1 to 18 wherein the or each protic acid is an inorganic acid.

20. A method as claimed in claim 19 wherein each protic acid is a hydrohalide acid.

21. A method as claimed in claim 19 wherein the or each protic acid is selected from HCl; HBr; HNO_{3;} H₂SO₄.

22. A method as claimed in any one of claims 1 to 18 wherein the or each protic acid is an organic acid.

23. A method as claimed in claim 22 wherein the or each protic acid is selected from H₂CO₃; CH₃COOH; methanesulfonic acid, 1,2-ethanedisulfonic acid, ethansulfonic acid, naphthalenedisulfonic acid, p-toluenesulfonic acid.

24. A method as claimed in any one of claims 1 to 18, or claim 23 wherein the MT-containing compound is LMTM:

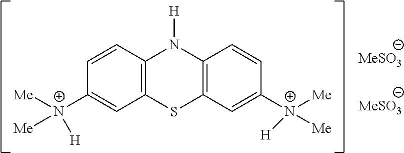

25. A method as claimed in claim 24 wherein the total daily dose of LMTM is at least 52 mg/day.

26. A method as claimed in claim 25 wherein the dose of LMTM is about 125 mg/once per day.

27. A method as claimed in any one of claims 1 to 18 wherein the MT-containing compound is selected from the list consisting of:

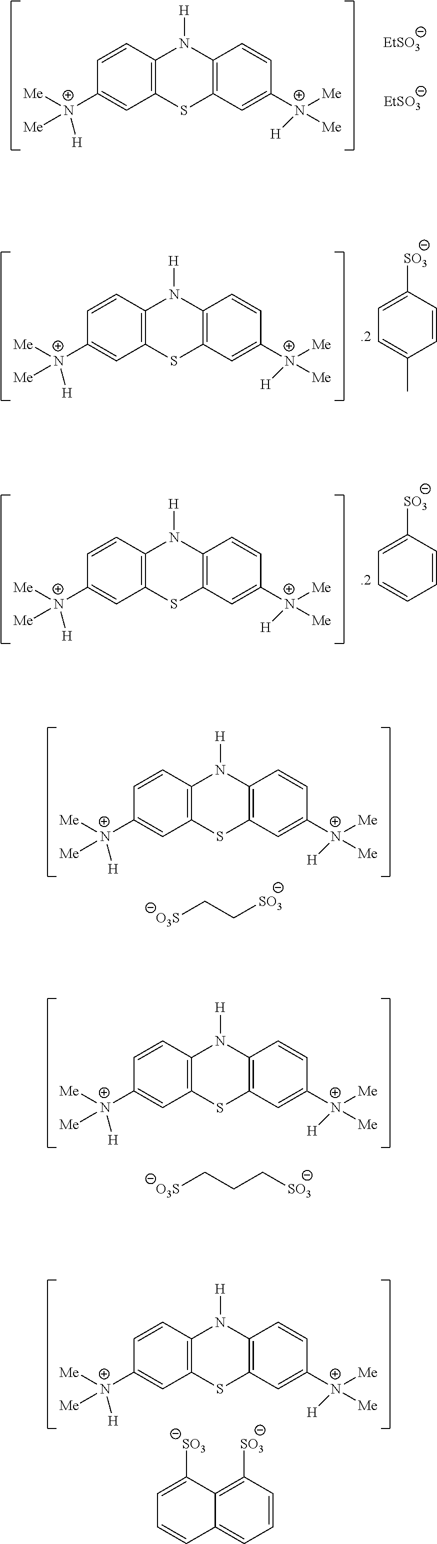

28. A method as claimed in any one of claims 1 to 27 wherein the treatment with the MT compound is such as to achieve one or more of the following in the subject:
(i) enhanced mitochondrial function;
(ii) enhanced blood oxygen capacity;
(iii) improved CNS sequelae arising from COVID-19.

29. An MT-containing compound as defined in any one of claims 1 to 28, for use in a method of treatment as defined in any one of claims 1 to 28.

30. Use of an MT-containing compound as defined in any one of claims 1 to 28, in the manufacture of a medicament for use in a method of treatment as defined in any one of claims 1 to 28.
